SEQUENCE LISTING (14 sequences)

SEQ ID NO: 1 - DNA, 66 bp, synthetic construct
  source 1..66: mol_type = other DNA; note = Erwinia carotovora
atgaaatacctgctgccgaccgctgctgctggtctgctgctcctcgctgcccagccggcgatggcc

SEQ ID NO: 2 - DNA, 63 bp, synthetic construct
  source 1..63: mol_type = other DNA; note = Escherichia coli Top10
atgaagcagagcaccatcgccctggccctgctgcccctgctgttcacccccgtgaccaaggcc

SEQ ID NO: 3 - DNA, 63 bp, synthetic construct
  source 1..63: mol_type = other DNA; note = Escherichia coli Top10
atgaagaagaccgccatcgccatcgccgtggccctggccggcttcgccaccgtggcccaggcc

SEQ ID NO: 4 - DNA, 462 bp, synthetic construct
  source 1..462: mol_type = other DNA; note = Sus scrofa f. domestica
atgggtctgagcgatggtgaatggcagctggttctgaatgtttggggtaaagttgaagcggatgtagctggccatggccaagaagtgctgattagactcttcaaaggtcatccggaaaccctggaaaaatttgataaattcaaacatctgaaatcagaagatgaaatgaaagcctcagaggacttgaagaaacacgggaacaccgtcttgacggctctaggggggatactcaagaagaagggacatcatgaagccgaattaacaccactcgcccaaagccatgcaaccaaacataaaatccccgtgaaatatctggaatttatctccgaggcaattattcaggttttacaatctaagcaccctggtgattttggtgccgacgcacagggtgcaatgtccaaggcgctcgaactgtttcgtaatgatatggcagcaaaatacaaagaactgggttttcagggc

SEQ ID NO: 5 - protein, 22 aa, synthetic construct
  source 1..22: note = Erwinia carotovora
MKYLLPTAAAGLLLLAAQPAMA

SEQ ID NO: 6 - protein, 21 aa, synthetic construct
  source 1..21: note = Escherichia coli Top10
MKQSTIALALLPLLFTPVTKA

SEQ ID NO: 7 - protein, 21 aa, synthetic construct
  source 1..21: note = Escherichia coli Top10
MKKTAIAIAVALAGFATVAQA

SEQ ID NO: 8 - protein, 154 aa, synthetic construct
  source 1..154: note = Sus scrofa f. domestica
MGLSDGEWQLVLNVWGKVEADVAGHGQEVLIRLFKGHPETLEKFDKFKHLKSEDEMKASEDLKKHGNTVLTALGGILKKKGHHEAELTPLAQSHATKHKIPVKYLEFISEAIIQVLQSKHPGDFGADAQGAMSKALELFRNDMAAKYKELGFQG

SEQ ID NO: 9 - DNA, 45 bp, synthetic construct
  source 1..45: mol_type = other DNA; note = Primer SsMB-F
caaggccatggctgatatcggatccatgggtctgagcgatggtga

SEQ ID NO: 10 - DNA, 42 bp, synthetic construct
  source 1..42: mol_type = other DNA; note = Primer SsMB-R
ggtggtggtggtggtgctcgagttagccctgaaaacccagtt

SEQ ID NO: 11 - DNA, 45 bp, synthetic construct
  source 1..45: mol_type = other DNA; note = Primer signal-F
gagcggataacaattcccctctagaaataattttgtttaacttta

SEQ ID NO: 12 - DNA, 42 bp, synthetic construct
  source 1..42: mol_type = other DNA; note = Primer SsMB-signal-R
ccttgtcgtcgtcgtcggtaccagaaccagaaccatgatgat

SEQ ID NO: 13 - DNA, 20 bp, synthetic construct
  source 1..20: mol_type = other DNA; note = Primer T7-F
taatacgactcactataggg

SEQ ID NO: 14 - DNA, 20 bp, synthetic construct
  source 1..20: mol_type = other DNA; note = Primer T7ter-R
tgctagttattgctcagcgg

(19) United States
(12) Patent Application Publication
(10) Pub. No.: US 2024/0287569 A1
(43) Pub. Date: Aug. 29, 2024

(54) MYOGLOBIN AND EXPRESSION VECTOR AND EXPRESSION ENGINEERING BACTERIUM THEREOF, AND USE THEREOF

(71) Applicants: Zhejiang University, Hangzhou City (CN); Ningbo Innovation Center, Zhejiang University, Ningbo City (CN); Shanghai Institute for Advanced Study of ZJU, Shanghai (CN)

(72) Inventors: Wei CHEN, Hangzhou City (CN); Lianghua XIE, Hangzhou City (CN); Yunhe MENG, Hangzhou City (CN); Kaihao YOU, Hangzhou City (CN); Jingwen ZHOU, Hangzhou City (CN); Xin GUAN, Hangzhou City (CN)

(21) Appl. No.: 18/405,235

(22) Filed: Jan. 5, 2024

(30) Foreign Application Priority Data
Feb. 23, 2023 (CN) 202310159430.7

(51) Int. Cl. C12P 21/02; C12N 9/16; C12N 9/88; C12N 15/70

(52) U.S. Cl. CPC C12P 21/02; C12N 9/16; C12N 9/88; C12N 15/70; C12Y 301/03001; C12Y 402/0201; C12N 2800/101

(57) ABSTRACT

The present disclosure provides myoglobin (MB) and an expression vector and an expression engineering bacterium thereof, and use thereof, and relates to the technical field of genetic engineering. In the present disclosure, recombinant Escherichia coli strains with signal peptides Pel B and Omp A inserted under same conditions have an expression level of Sus scrofa myoglobin (SsMB) increased by 2.06 and 1.17 times, respectively, compared with an original expression strain of the SsMB. The signal peptides that can increase the expression level of the SsMB provide a new idea for research and application of improving the expression level of the SsMB.

### CROSS REFERENCE TO RELATED APPLICATION

This patent application claims the benefit and priority of Chinese Patent Application No. 202310159430.7 filed with the China National Intellectual Property Administration on Feb. 23, 2023, the disclosure of which is incorporated by reference herein in its entirety as part of the present application.

### REFERENCE TO SEQUENCE LISTING

A computer readable XML file entitled “SEQUENCE_LISTING”, that was created on Nov. 23, 2023, with a file size of about 16516 bytes, contains the sequence listing for this application, has been filed with this application, and is hereby incorporated by reference in its entirety.

### TECHNICAL FIELD

The present disclosure relates to the technical field of genetic engineering, and relates to myoglobin and an expression vector and an expression engineering bacterium thereof, and use thereof.

### BACKGROUND

Artificial meat is a type of edible meat produced without using traditional livestock and poultry breeding methods. The emergence of artificial meat provides new ideas for the current tense meat production and provides a solution to alleviate the imbalanced supply relationship of meat. As one of the main sensory indicators, color can directly affect consumers' purchasing and consumption desires. However, artificial meat products currently available cannot produce a color similar to real meat due to low heme content. To improve the appearance of artificial meat, some food additives are used in artificial meat to imitate the coloring of real meat. For example, exogenous addition of myoglobin (MB) is conducted to simulate the color of real meat.

Although the MB can be obtained directly from animals, the operation method not only wastes time and energy, but also further increases a production cost of the artificial meat. Therefore, animal-free and biotechnological production of MB is essential for high-yielding, economical, and environmental-friendly artificial meat production. At present, there have been many reports on biosynthetic MB research at home and abroad. This cost-effective production method has laid the foundation for the application of MB in artificial meat. However, there are still many limitations in the biosynthesis of MB, with a yield to be further improved.

MB is found in bone and heart tissue and is one of the heme-binding proteins. MB is structurally similar to the a subunit of hemoglobin and serves as an intracellular oxygen reservoir. Due to catalytic and spectral properties, the MB is widely used in biocatalysts, iron supplements, and disease diagnosis. Especially in recent years, since it is closely related to the formation of meat color and the metallic taste of meat, the MB has a potential to be used as a color additive for plant-based and cell-based artificial meat. MB and hemoglobin in animal meat tissues are important sources of dietary iron for humans. In animal-derived meat, the MB unfolds upon cooking and exposes a heme cofactor, which then catalyzes a series of chemical reactions. These chemical reactions convert naturally occurring amino acids, nucleotides, vitamins, and sugars in animal muscle tissue into hundreds of highly-specific and diverse flavor and aroma compounds, resulting in the distinctive and unique flavor of meat.

At present, MB is mainly extracted from animal cardiac muscle, skeletal muscle and other muscle tissues. However, there are also challenges such as the rapid spontaneous oxidation of the MB extracted from muscle samples and the limited supply of the MB due to this extraction method, making large-scale production difficult. From the perspective of sustainable development, manufacturing MB based on microbial culture technology to develop “artificial meat” solves the unsustainable problems of food raw materials and production methods. Moreover, this technology is economical- and environmental-friendly, showing a great application potential and a scientific research value. In view of this, the production of MB using microbial fermentation technology has attracted widespread attention and exhibits great market prospects.

### SUMMARY

Based on the deficiencies in the prior art, the present disclosure provides myoglobin (MB) and an expression vector and an expression engineering bacterium thereof, and use thereof. The efficient expression and secretion of Sus scrofa myoglobin (SsMB) in Escherichia coli is achieved by introducing two signal peptides, Pel B and Omp A.

In the present disclosure, a signal peptide of pectin lyase Pel B from Erwinia carotovora, a signal peptide of alkaline phosphatase Pho A that regulates metabolism and transport of Escherichia coli, and a signal peptide of outer membrane protein Omp A of the Escherichia coli are inserted into a recombinant plasmid expressing SsMB. Compared with an expression plasmid of the original SsMB, the expression levels of SsMB of the three signal peptides are increased by 2.06, 0.58, and 1.17 times, respectively. The Pel B signal peptide and Omp A signal peptide better solve the problem of low SsMB protein expression and lay the foundation for the biosynthesis of SsMB and the simulation of artificial meat.

The present disclosure adopts the following technical solutions:

The present disclosure provides a gene expression cassette encoding MB, including an encoding gene of a signal peptide and an encoding gene of domestic pig (Sus scrofa f. domestica)-derived MB (SsMB) sequentially; where the signal peptide is selected from the group consisting of a signal peptide Pel B and a signal peptide Omp A.

Specifically, the MB has an encoding gene sequence shown in SEQ ID NO: 4, the signal peptide Pel B has an encoding gene sequence shown in SEQ ID NO: 1, and the signal peptide Omp A has an encoding gene sequence shown in SEQ ID NO: 3.

The present disclosure further provides a plasmid comprising the gene expression cassette encoding MB.

In an embodiment of the present disclosure, the plasmid has an expression vector pET-32a.

A construction process of the plasmid includes the following steps:
subjecting a target gene of SsMB and the pET-32a plasmid to double digestion by BamH I and Xho I endonucleases, ligating to obtain a pET-32a-SsMB expression plasmid, and sequencing to verify a correctness of the target gene;
extracting an exogenous genome of an Escherichia coli TOP10 strain, amplifying Pel B and Omp A genes by PCP using a primer signal-F and a primer SsMB-signal-R with a pUC57 genome as a template, respectively; and
subjecting the pET-32a-SsMB expression plasmid to double digestion with Xba I and Kpn I, purifying, ligating the signal peptide genes and the vector with a homologous recombinase to obtain recombinant plasmids pET-32a-Pel B-SsMB and pET-32a-Omp A-SsMB that have the Pel B and Omp A signal peptides, respectively, and conducting colony PCR and sequencing to verify the recombinant plasmids.

The present disclosure further provides a cell including the plasmid.

In an embodiment of the present disclosure, the cell is Escherichia coli BL21 (DE3) cell. The present disclosure further provides use of the cell in preparation of MB.

The present disclosure further provides a protein expression method, including the following steps: inoculating the cell into an LB medium to allow culture until an OD₆₀₀ value is 0.6, adding isopropyl-β-d-thiogalactoside (IPTG) with a final concentration of 0.01 mM to 1 mM, and conducting induction at 16° C. to 37° C. and 180 rpm for 8 h to 20 h.

Preferably, the IPTG at a final concentration of 0.1 mM is added to allow the induction at 20° C. and 180 rpm for 12 h.

SsMB engineering strains expressing different signal peptides are cultured separately, and then subjected to induced expression according to the above conditions to screen and express the engineering strains, and a protein expression level is detected by SDS-PAGE.

The present disclosure has following beneficial effects:

1. In the present disclosure, recombinant Escherichia coli strains with signal peptides Pel B and Omp A inserted under same conditions have an expression level of SsMB increased by 2.06 and 1.17 times, respectively, compared with an original expression strain of the SsMB.

2. In the present disclosure, the signal peptides that can increase the expression level of the SsMB provide a new idea for research and application of improving the expression level of the SsMB.

### BRIEF DESCRIPTION OF THE DRAWINGS

FIG. 1 shows a plasmid map of pET-32a-SsMB;

FIG. 2 shows a plasmid map of pET-32a-Pel B-SsMB;

FIG. 3 shows a plasmid map of pET-32a-Pho A-SsMB;

FIG. 4 shows a plasmid map of pET-32a-Omp A-SsMB;

FIG. 5 shows verification results of fragment sizes of the three signal peptides Pel B, Pho A, and Omp A; where Lane 1: 100 bp DNA Ladder, Lanes 2 and 3: Pel B amplified fragments, Lanes 4 and 5: Pho A amplified fragments, Lanes 6 and 7: Omp A amplified fragments;

FIG. 6 shows verification results of plate colony PCR on transforming BL21 (DE3) with the pET-32a-Pel B-SsMB recombinant plasmid; where Lanes 1 and 14: 100 bp DNA Ladder, Lanes 2 and 13: negative control, Lanes 3 to 12: verification results of plate colony PCR of pET-32a-Pel B-SsMB transformation;

FIG. 7 shows verification results of plate colony PCR on transforming BL21 (DE3) with the pET-32a-Pho A-SsMB recombinant plasmid; where Lanes 1 and 14: 100 bp DNA Ladder, Lanes 2 and 13: negative control, Lanes 3 to 12: verification results of plate colony PCR of pET-32a-Pho A-SsMB transformation;

FIG. 8 shows verification results of plate colony PCR on transforming BL21 (DE3) with the pET-32a-Omp A-SsMB recombinant plasmid; where Lanes 1 and 14: 100 bp DNA Ladder, Lanes 2 and 13: negative control, Lanes 3 to 12: verification results of plate colony PCR of pET-32a-Omp A-SsMB transformation;

FIG. 9 shows induced expression results of SsMB with different signal peptides; where Lane 1: protein marker; Lane 2: expression of SsMB protein without signal peptide; Lane 3: expression of SsMB protein with Pel B signal peptide; Lane 4: expression of SsMB protein with Pho A signal peptide; Lane 5: expression of SsMB protein with Omp A signal peptide;

FIG. 10 shows relative expression levels of SsMB in strains with different signal peptides; ****p<0.0001;

FIG. 11 shows an influence of IPTG with different concentrations on an expression level of the target protein; where Lane 1: protein marker; Lane 2: SsMB protein expression when induced by 0.01 mM IPTG; Lane 3: SsMB protein expression when induced by 0.02 mM IPTG; Lane 4: SsMB protein expression when induced by 0.05 mM IPTG; Lane 5: SsMB protein expression when induced by 0.1 mM IPTG; Lane 6: SsMB protein expression when induced by 0.2 mM IPTG; Lane 7: SsMB protein expression when induced by 0.5 mM IPTG; Lane 8: SsMB protein expression when induced by 1 mM IPTG;

FIG. 12 shows an influence of different induction temperatures on the expression level of the target protein; where Lane 1: protein marker; Lane 2: SsMB protein expression when induced at 16° C.; Lane 3: SsMB protein expression when induced at 20° C.; Lane 4: SsMB protein expression when induced at 25° C.; Lane 5: SsMB protein expression when induced at 30° C.; Lane 6: SsMB protein expression when induced at 34° C.; Lane 7: SsMB protein expression when induced at 37° C.; and

FIG. 13 shows an influence of different induction times on the expression level of the target protein; where Lane 1: protein marker; Lane 2: SsMB protein expression at 8 h of induction; Lane 3: SsMB protein expression at 12 h of induction; Lane 4: SsMB protein expression at 16 h of induction; Lane 5: SsMB protein expression at 20 h of induction.

### DETAILED DESCRIPTION OF THE EMBODIMENTS

LB liquid medium: 10 g/L tryptone, 5 g/L yeast extract, 10 g/L NaCl, sterilized at 121° C. for 20 min, for the culturing of Escherichia coli.

LB solid medium: 20 g/L agar powder added to the LB liquid medium, for the culturing and screening of Escherichia coli.

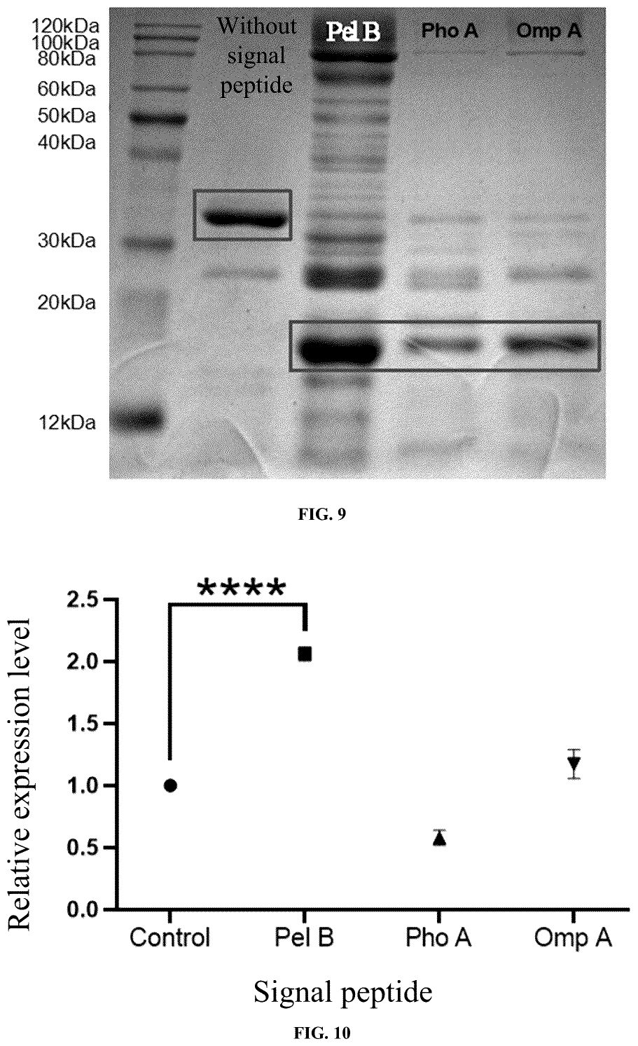

In the present disclosure, the SsMB proteins with different signal peptides appear as bands at expected positions separately, and a molecular weight of the protein is consistent with a theoretical value. FIG. 9 shows a comparison of the expression levels of SsMB proteins with different signal peptides under the same induction conditions; FIG. 10 shows the relative expression levels of SsMB proteins with different signal peptides. Specific situations and results are given in the following examples.

### Example 1

1. Construction of pET-32a-SsMB Vector

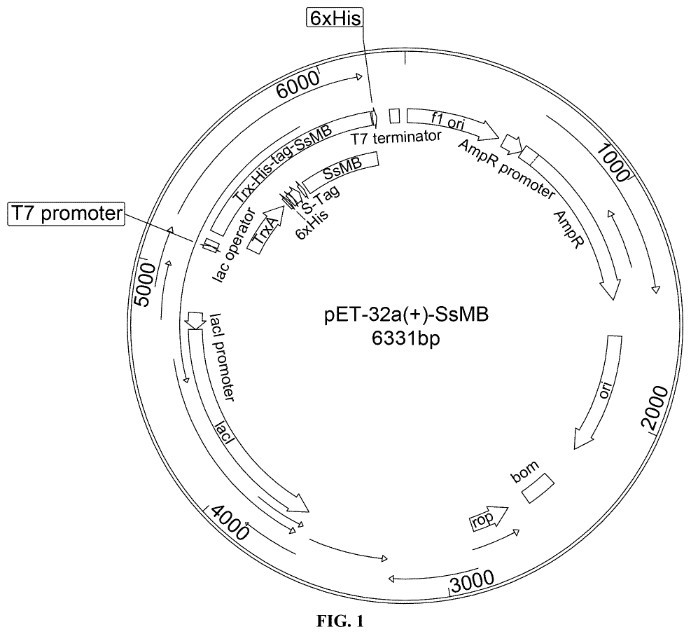

A gene sequence of SsMB was obtained from NCBI (Gene ID: 397467, a gene sequence was shown in SEQ ID NO: 4), and then synthesized by Tsingke Biotech Co., Ltd., and PCR was conducted using primers SsMB-F and SsMB-R in Table 2 to amplify the target gene. A pET-32a empty vector was double-digested using BamH I and Xho I enzymes (Takara, Dalian), and then the synthesized gene was ligated to the digested pET-32a vector using a homologous recombinase Trelief® Seamless Cloning Kit (Tsingke Biotech, Hangzhou) to construct a recombinant expression vector pET-32a-SsMB of SsMB. A spectrum of the recombinant expression vector was shown in FIG. 1.

### 2. Construction of Recombinant Plasmid Expression Vector

Step S1: an exogenous genome of E. coli Top10 strain (Tsingke Biotech, Hangzhou) was extracted using Yeasen kit; PCR was conducted using a pUC57 genome as a template and primers signal-F and SsMB-signal-R in Table 2 to amplify genes encoding Pel B, Pho A, and Omp A; a size of the target gene fragment was identified by agarose gel electrophoresis (FIG. 5), and amino acid sequences and encoding gene sequences of the signal peptides Pel B, Pho A, and Omp A were shown in Table 1.

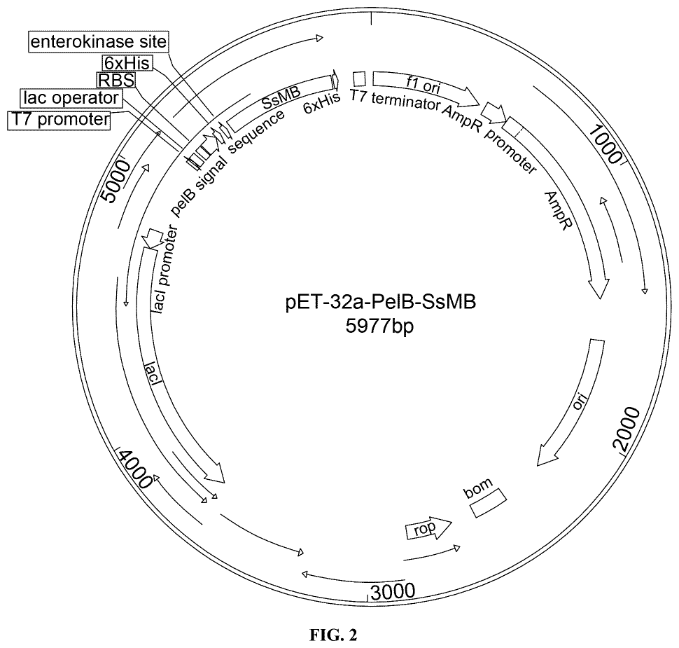

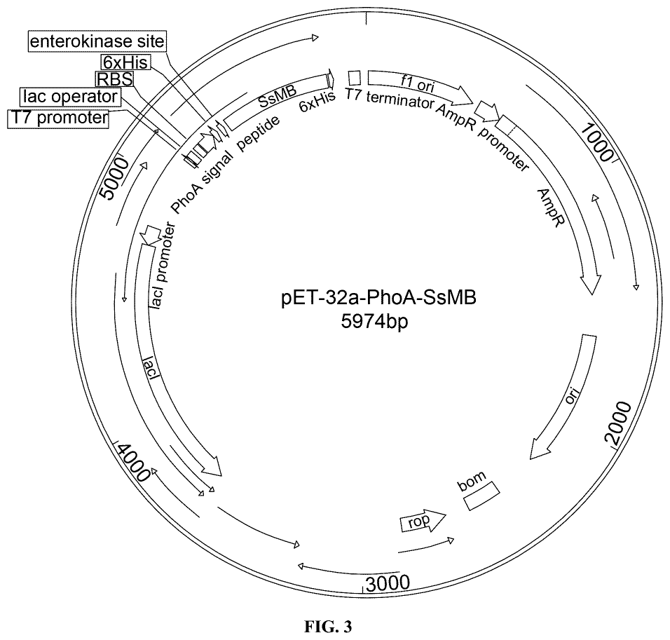

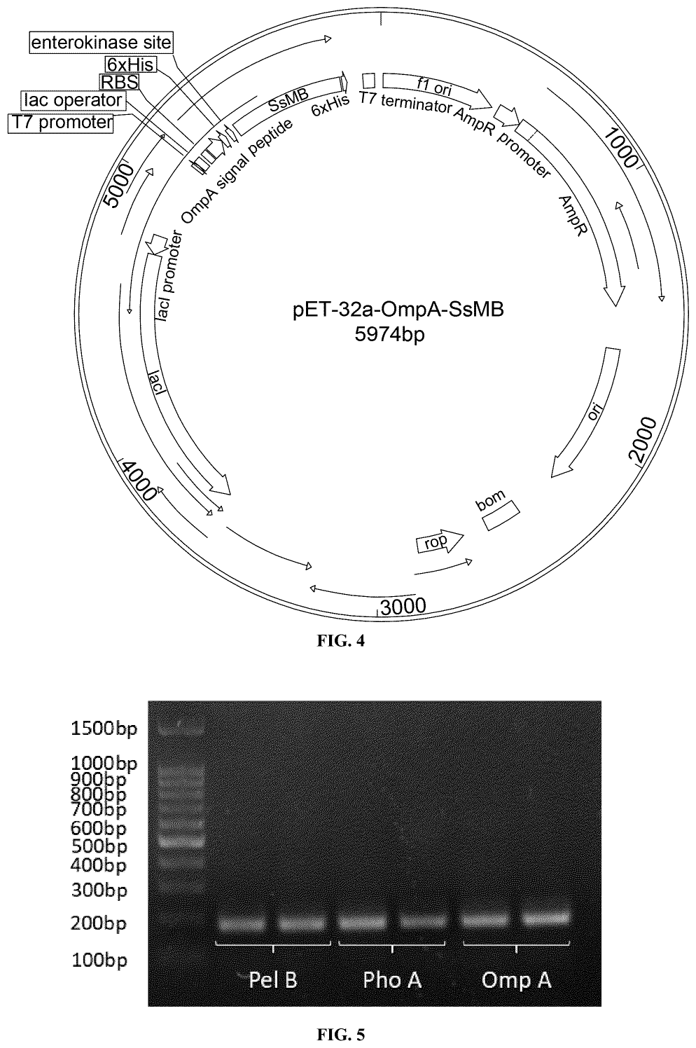

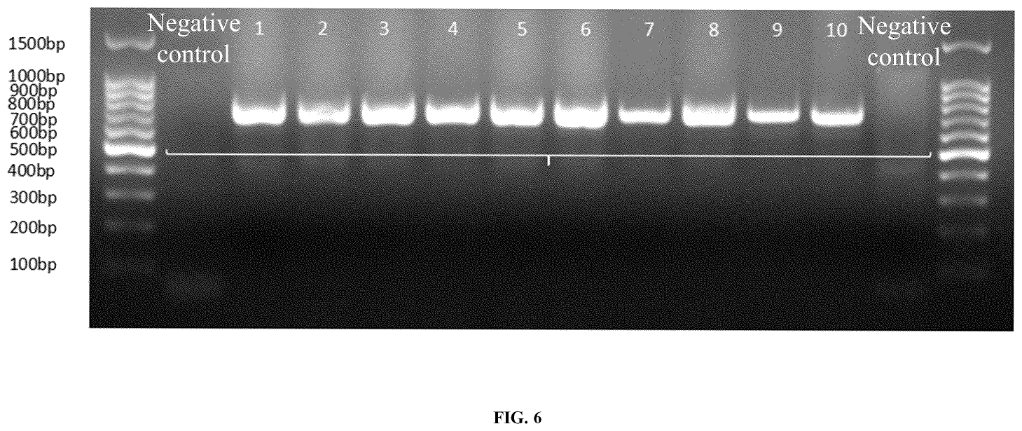

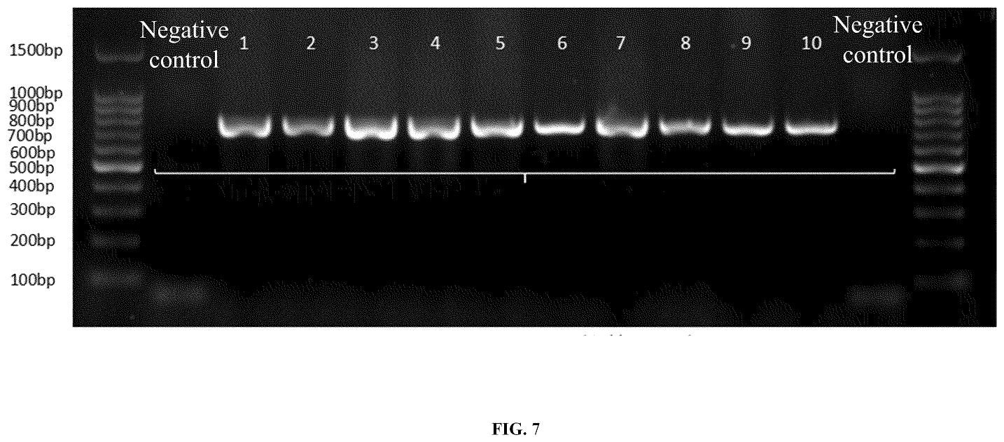

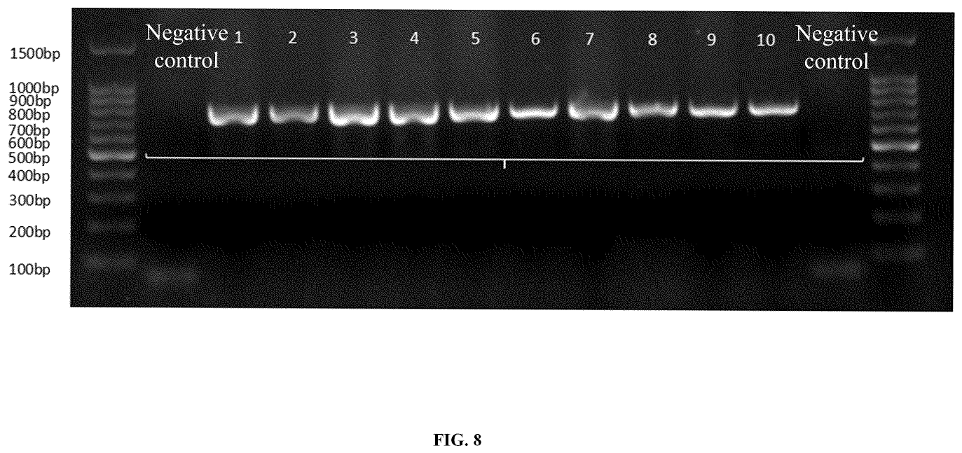

Step S2: the pET-32a-SsMB plasmid was double-digested using Xba I and Kpn I enzymes (Takara, Dalian), and then purified; the signal peptide encoding genes (encoding Pel B, Pho A, and Omp A genes) obtained in step S1 and the digested vector pET-32a-SsMB were ligated with a homologous recombinase Trelief® Seamless Cloning Kit, to obtain pET-32a-Pel B-SsMB (with a map shown in FIG. 2), pET-32a-Pho A-SsMB (with a map shown in FIG. 3), and pET-32a-Omp A-SsMB plasmid (with a map shown in FIG. 4) containing gene sequences encoding the Pel B, Pho A, and Omp A signal peptides; the plasmids were transformed into E. coli BL21 (DE3) competent cells, spread on a LB solid medium containing 1‰ of 100 μg/mL ampicillin, and then incubated overnight at 37° C. 10 single colonies on each plate were put in 10 μL sterilized water, PCR and sequencing were conducted separately using obtained bacterial solutions of the 10 single clones as a template, T7-F in Table 2 as an upstream primer, and T7ter-R as a downstream primer to verify the constructed recombinant plasmid (the negative control used sterilized water as a template). The colony PCR verification results were shown in FIG. 6 to FIG. 8. The single colonies selected from the transformation plates of the three recombinant plasmids containing signal peptides all showed bands at the expected positions, while the negative control had no bands at the corresponding positions. This indicated that the three recombinant plasmids containing signal peptides were successfully constructed and transformed into the E. coli BL21(DE3) competent cells.

TABLE 1
Amino acid and base sequences of
different signal peptides

| | | Amino acid | Nucleotide |
|---|---|---|---|
| Name | kDa | sequence | sequence |
| Pel B | 2.23 | MKYLLPTAAAGL | 5′-ATGAAATACCTGCT |
| | | LLLAAQPAMA | GCCGACCGCTGCTGCTG |
| | | (SEQ ID NO: 5) | GTCTGCTGCTCCTCGCT |
| | | | GCCCAGCCGGCGATGGC |
| | | | C-3′ |
| | | | (SEQ ID NO: 1) |
| Pho A | 2.26 | MKQSTIALALL | 5′-ATGAAGCAGAGCAC |
| | | PLLFTPVTKA | CATCGCCCTGGCCCTGC |
| | | (SEQ ID NO: 6) | TGCCCCTGCTGTTCACC |
| | | | CCCGTGACCAAGGCC-3′ |
| | | | (SEQ ID NO: 2) |
| Omp A | 2.05 | MKKTAIAIAVA | 5′-ATGAAGAAGACCGC |
| | | LAGFATVAQA | CATCGCCATCGCCGTGG |
| | | (SEQ ID NO: 7) | CCCTGGCCGGCTTCGCC |
| | | | ACCGTGGCCCAGGCC-3′ |
| | | | (SEQ ID NO: 3) |

TABLE 2
Primer sequences for constructing vectors

| | | Restriction | |
|---|---|---|---|
| | | site | |
| Primer | | (underlined | |
| name | Primer sequence | part) | Use |
| SsMB-F | 5′-CAAGGCCATGGCTGATAT | BamH I | Upstream primer for |
| | CGGATCCATGGGTCTGAGCG | | SsMB amplification |
| | ATGGTGA-3′ (SEQ ID NO: 9) | | |
| SsMB-R | 5′-GGTGGTGGTGGTGGTGCT | Xho I | Downstream primer |
| | CGAGTTAGCCCTGAAAACCC | | for SsMB |
| | AGTT-3′ (SEQ ID NO: 10) | | amplification |
| signal-F | 5′-GAGCGGATAACAATTCCC | Xba I | Upstream primers |
| | CTCTAGAAATAATTTTGTTTA | | for amplification of |
| | ACTTTA-3′ (SEQ ID NO: 11) | | three signal peptides |
| SsMB-signal- | 5′-CCTTGTCGTCGTCGTCG | Kpn I | Downstream |
| R | GTACCAGAACCAGAACCATGA | | primers for |
| | TGAT-3′ (SEQ ID NO: 12) | | amplification of |
| | | | three signal peptides |
| T7-F | 5′-TAATACGACTCACTATAG | — | Upstream primers |
| | GG-3′ | | for colony PCR |
| | (SEQ ID NO: 13) | | validation |
| T7ter-R | 5′-TGCTAGTTATTGCTCAGC | — | Downstream |
| | GG-3′ (SEQ ID NO: 14) | | primers for colony |
| | | | PCR validation |

### Example 2

### 1. Optimization of Induced Expression Conditions

1.1 Influence of IPTG with Different Concentrations on the Expression Level of Target Protein

The SsMB target gene sequence was ligated to the pET-32a vector. For specific steps, see step 1 in Example 1. The plasmid was transformed into BL21 (DE3) competent cells for expression to determine the SsMB induced expression conditions. A single colony of E. coli that was successfully transformed and sequenced correctly was inoculated into a 5 mL liquid LB test tube containing ampicillin, and then cultured overnight at 37° C. and 220 rpm. A resulting bacterial solution cultured overnight was inoculated into 100 mL of ampicillin-resistant LB liquid medium at an inoculum volume of 1%, cultured at 37° C. and 220 rpm, and an OD value was detected; at a time point when the OD₆₀₀ value was 0.6, IPTG with final concentrations of 0.01 mM, 0.02 mM, 0.05 mM, 0.1 mM, 0.2 mM, 0.5 mM, and 1 mM were added separately, and induced for 20 h at 20° C. and 180 rpm; SDS-PAGE was conducted to compare the influence of IPTG with final concentrations on the expression level of the target protein (the results were shown in FIG. 11), thereby determining an optimal IPTG concentration.

### 1.2 Influence of Different Induction Temperatures on the Expression Level of Target Protein

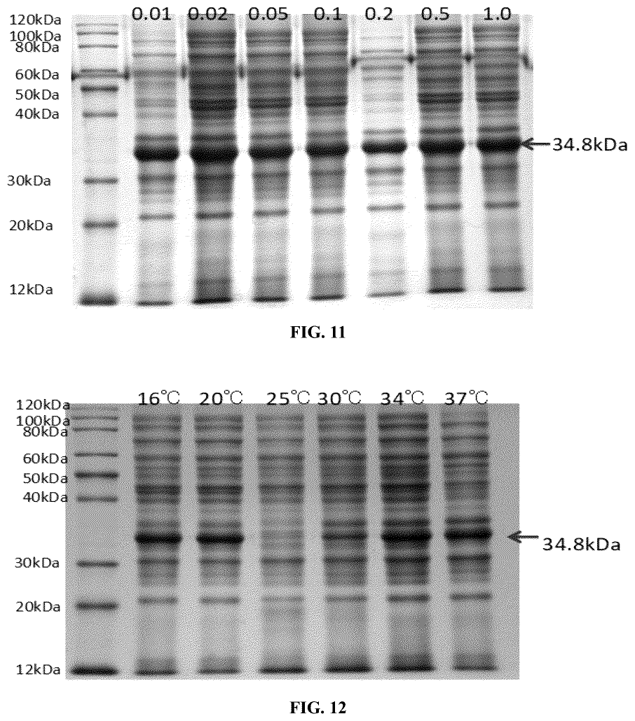

An induction temperature was adjusted according to the optimal IPTG concentration. A single colony of E. coli that was successfully transformed and sequenced correctly was inoculated into a 5 mL liquid LB test tube containing ampicillin, and then cultured overnight at 37° C. and 220 rpm. A resulting bacterial solution cultured overnight was inoculated into 100 mL of ampicillin-resistant LB liquid medium at an inoculum volume of 1%, cultured at 37° C. and 220 rpm, and an OD value was detected; at a time point when the OD₆₀₀ value was 0.6, an appropriate concentration of IPTG was added and then induced at 16° C., 20° C., 25° C., 30° C., 34° C., and 37° C. and 180 rpm for 20 h; SDS-PAGE was conducted to compare the influence of IPTG with final concentrations on the expression level of the target protein (the results were shown in FIG. 12), thereby determining an optimal induction temperature.

### 1.3 Influence of Different Induction Times on the Expression Level of Target Protein

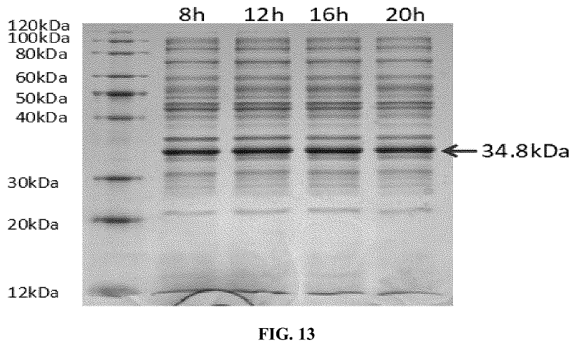

An induction time was adjusted according to the optimal IPTG concentration and optimal induction temperature. A single colony of E. coli that was successfully transformed and sequenced correctly was inoculated into a 5 mL liquid LB test tube containing ampicillin, and then cultured overnight at 37° C. and 220 rpm. A resulting bacterial solution cultured overnight was inoculated into 100 mL of ampicillin-resistant LB liquid medium at an inoculum volume of 1%, cultured at 37° C. and 220 rpm, and an OD value was detected; at a time point when the OD₆₀₀ value was 0.6, an appropriate concentration of IPTG was added and then induced at 20° C. and 180 rpm for 8 h, 12 h, 16 h, and 20 h; SDS-PAGE was conducted to compare the influence of IPTG with final concentrations on the expression level of the target protein (the results were shown in FIG. 13), thereby determining an optimal induction time.

According to the optimization results of induced expression, the expression conditions of SsMB protein without signal peptide were optimized. The best induction conditions were obtained under the condition of OD₆₀₀=0.6 by adding IPTG with a final concentration of 0.01 mM and inducing at 20° C. and 180 rpm for 12 h.

2. Induced Expression of SsMB Protein Containing Signal Peptide in E. coli

The expression vectors pET-32a-Pel B-SsMB, pET-32a-Pho A-SsMB, and pET-32a-Omp A-SsMB constructed in Example 1 and containing signal peptides were transformed into DH5a E. coli competent cells, respectively. Transformants were selected for sequencing verification, and plasmid extraction was conducted after successful verification. The three SsMB protein expression plasmids with different signal peptides were transformed into BL21 (DE3) E. coli competent cells, and the transformants were selected for sequencing verification. After successful verification, induced expression was conducted. Transformation system: 5 μL of recombinant plasmid and 100 μL of competent cells were mixed; a resulting mixed system was placed on ice for 30 min, heat-shocked at 42° C. for 80 s. After the heat shock was completed, the mixed system was placed on ice for 2 min, and then 700 μL of LB medium without resistance was added and cultured at 37° C. for 1 h. The mixed system was centrifuged at 4,000 rpm for 3 min, the supernatant was discarded, and the pellet was resuspended in 200 μL of sterilized water, then spread on LB solid medium containing 1‰ of 100 μg/mL ampicillin, and incubated overnight at 37° C. The next day, single clone transformants were picked for verification.

Induced expression: induced expression was conducted according to the induced expression optimization results in step 1.

The correctly sequenced monoclonal bacterial solution was inoculated into 5 mL LB liquid medium containing 1‰ of 100 μg/mL ampicillin, and cultured overnight at 37° C. and 220 rpm. The bacterial solution cultured overnight was inoculated into 100 mL liquid LB medium containing 1‰ of 100 μg/mL ampicillin at an inoculum volume of 1%, and cultured at 37° C. and 220 rpm until OD₆₀₀=0.6. The IPTG with a final concentration 0.01 mM was added, and low-temperature induction was conducted at 20° C. and 180 rpm for 12 h.

### 3. His-Tag Protein Purification

Preliminary purification was conducted on the protein in the induction supernatant using HisTALON™ Gravity Column.

The induced bacterial solution was centrifuged at 4° C. and 4,000 rpm for 30 min, and a supernatant was discarded. The centrifuged pellet was resuspended in PBS and then disrupted by sonication. The disrupted sample was centrifuged at 4° C. and 4,000 rpm for 30 min, and a resulting supernatant was passed through a 0.45 μm aqueous filter membrane. The protein in the induction supernatant was subjected to preliminary column purification using the purification steps of HisTALON™ Gravity Column.

Protein expression levels were identified by SDS-PAGE, and relative quantification of the protein expression was conducted using Image J. The target protein was quantified using the Shenhua gel imaging system, and the relative expression level of SsMB without signal peptide was set as 1.00. Accordingly, the relative expression level of SsMB with Pel B signal peptide was 2.06, the relative expression level of SsMB with Pho A signal peptide was 0.58, and the relative expression level of SsMB with Omp A signal peptide was 1.17 (FIG. 9 and FIG. 10). These results showed that among the three signal peptides of the present disclosure, Pel B and Omp A could increase the expression level of SsMB to varying degrees, and the increased protein expression levels from high to low were: Pel B>Omp A.

In the present disclosure, after the Pel B signal peptide was inserted into the SsMB recombinant expression vector, the expression level of SsMB was increased by 2.05 times compared with that without the signal peptide; after the Omp A signal peptide was inserted into the SsMB recombinant expression vector, the expression level of SsMB was increased by 1.17 times compared with that without the signal peptide.

1. A gene expression cassette encoding myoglobin (MB), comprising an encoding gene of a signal peptide and an encoding gene of domestic pig (Sus scrofa f. domestica)-derived MB (SsMB) sequentially; wherein the signal peptide is selected from the group consisting of a signal peptide Pel B and a signal peptide Omp A.

2. The gene expression cassette encoding MB according to claim 1, wherein the MB has an encoding gene sequence shown in SEQ ID NO: 4, the signal peptide Pel B has an encoding gene sequence shown in SEQ ID NO: 1, and the signal peptide Omp A has an encoding gene sequence shown in SEQ ID NO: 3.

3-4. (canceled)

5. A cell comprising a plasmid comprising the gene expression cassette encoding MB according to claim 1.

6. The cell according to claim 5, wherein the cell is an Escherichia coli BL21 (DE3) cell.

7. (canceled)

8. A protein expression method, comprising the following steps: inoculating the cell according to claim 5 into an LB medium to allow culture until an OD₆₀₀ value is 0.6, adding isopropyl-β-d-thiogalactoside (IPTG) with a final concentration of 0.01 mM to 1 mM, and conducting induction at 16° C. to 37° C. and 180 rpm for 8 h to 20 h.

9. The protein expression method according to claim 8, wherein the IPTG at a final concentration of 0.01 mM is added to allow the induction at 20° C. and 180 rpm for 12 h.

10. The cell according to claim 5, wherein the MB has an encoding gene sequence shown in SEQ ID NO: 4, the signal peptide PeI B has an encoding gene sequence shown in SEQ ID NO: 1, and the signal peptide Omp A has an encoding gene sequence shown in SEQ ID NO: 3.

11. The cell according to claim 5, wherein the plasmid further comprises an expression vector pET-32a.

12. (canceled)
